(19) United States
(12) Patent Application Publication
(10) Pub. No.: US 2003/0220663 A1
(43) Pub. Date: Nov. 27, 2003

(54) Lancet device

(76) Inventors: Henry H. Fletcher, Cameron Park, CA (US); Ratan K. Bajaj, Dublin, CA (US); Scott T. Kaminski, San Ramon, CA (US)

Correspondence Address: GEORGE W. WASSON, 3123 INDIAN WAY, LAFAYETTE, CA, 94549, US

(21) Appl. No.: 10/154,087

(22) Filed: May 22, 2002

(51) Int. Cl. A61B 17/14

(52) U.S. Cl. 606/182

(57) ABSTRACT

A lancet device for drawing blood from a digit of a patient. The lancet device is particularly directed to a disposable, pre-armed or armable device including an element for constricting the finger or digit of a patient or user from which the blood is to be drawn. The device includes a portion that functions in a tourniquet manner to increase the blood supply in the area where the lancet is to create an incision. The lancet portion and the structure of the device are designed to produce a substantially painless incision in the patient. The biasing elements of the device provide for forcing the lancet portion toward the target area and for retrieving the lancet into the device after an incision has been made. After use in making an incision, the parts of the device that contact the patient's blood are withdrawn from possible inadvertent contact thus avoiding a chance of contamination of the user with the patient's blood.

This invention relates to the field of blood-sampling devices, particularly to a disposable, pre-armed or armable lancet device capable of constricting the finger from which the blood is to be drawn.

For purposes of medical diagnosis it is often necessary for patients to provide small specimens of blood to be analyzed. When it is required that such analysis be performed on a routine basis, as is often the case with patients suffering from diabetes or those diagnosed with the Human Immunodeficiency Virus, patients may have to obtain blood specimens without the help of a nurse or other assistant. Several disposable apparatus allowing patients to collect blood samples without the aid of others are available. Since all such devices obtain the blood by producing a small incision in the skin of the patient (most commonly the skin of one of the digits of the hand) with a sharp lancet, it is imperative that devices of this type be executed in a manner which allows them to be safely disposed after use without risking injury and possible infection to the person who may come into contact with these devices after their disposal. Another desirable feature of instruments for collection of blood samples is the inability of the patient to anticipate the incision and so attempt to withdraw therefrom.

Even though the makers of known blood-sampling devices have attempted to address the above-mentioned concerns by providing safer and more convienient instruments, prior-art units continue to fall short in several areas. Significantly, prior-art units require the patient (or the assistant to either manually squeeze the blood from the incision or to employ a separate tourniquet for this purpose, thus complicating the procedure and making it more stressful for the patient. Additionally, if the act of squeezing the blood from the incision is manually performed by an assistant who massages the skin around the incision, the contact with the blood of the patient, even when made by the assistant's hands protected with surgical gloves, presents the assistant with a risk of infection. Furthermore, obtaining a blood sample from the patient's finger using a conventional lancet device often causes the patient to experience a painful sensation. This occurs when the incision in one of the digits of the patient's hand is made so that the lancet strikes too close to the bone (e.g. the distal phalanx) of the digit.

### BRIEF SUMMARY OF THE INVENTION

It is accordingly desirable to provide a lancet device that operates in a manner to minimize the pain caused by the incision.

It is desirable to provide a lancet device that increases the efficiency with which blood is collected from the incision without unnecessary discomfort to the patient or additional risk of infection to the assistant gathering the blood sample, if such an assistant is utilized.

Because lancets and their associated parts are usually considered to be a disposable unit used one time, it is further desirable that the lancet and its associated parts and be easily assembly be inexpensively manufactured.

Because of the chance of exposure of the user or an assistant to infectious materials, it is desirable that the part of the lancet that contacts such infectious material be withdrawn from open exposure after an incision has been created with the lancet.

It is desirable that the lancet assembly accomplish a contact with a patient's digit in a manner that will operate to increase blood supply to the target area where an incision is to be created.

Further objects and features of the present invention will be readily apparent to those skilled in the art from the appended drawings and specification illustrating preferred embodiments.

In the embodiments of the invention illustrated herein, the lancet device comprises an adjustable digit-constricting member that is movably and operatively coupled to an outer sleeve. An inner sleeve is movably disposed relative to the outer sleeve. A lanceted plunger is movable within the outer sleeve with the inner sleeve interposed between the outer sleeve and the lanceted plunger. The lanceted plunger is releasably engaged within the inner sleeve and a biasing element biases the lanceted plunger relative to the inner sleeve.

### BRIEF DESCRIPTION OF THE SEVERAL VIEWS OF THE DRAWINGS

FIG. 1 is an exploded perspective of a lancet device in accordance with one embodiment of the present invention.

FIG. 2 is a sectional view of the outer sleeve of FIG. 1.

FIG. 3 is an assembly of the outer sleeve, inner sleeve and lanceted plunger of FIG. 1

FIG. 4 is a sectional view of the hollow cylindrical body of FIG. 1.

FIG. 5 is a perspective view of the clamping member of the lancet device.

FIG. 6 is a sectional view through the clamping member of FIGS. 5 and 7 taken along the lines 6-6 of FIG. 7.

FIG. 7 is a bottom plan view of the clamping member of FIG. 5.

FIG. 8 is a perspective view of the assembled and armed lancet device of the embodiment of FIG. 1.

FIG. 9 is a sectional view of an aternative embodiment of the invention.

FIG. 10 is an alternative form of the biasing means for biasing the outer sleeve with respect to the inner sleeve.

FIG. 11 is an illustration of the flex position for the biasing means of FIG. 10.

FIG. 12 is a sectional view of another alternative form of the invention.

FIG. 14 illustrates the retracted position of the lancet in the embodiment of FIG. 12 after an incision has been made.

For purposes of illustration, these figures are not necessarily drawn to scale. In all of the figures, same components are designated by the same reference numerals.

### DETAILED DESCRIPTION OF THE INVENTION

Throughout the following description, specific details are set forth in order to provide a more thorough understanding of the invention. However, the invention may be practiced without these particulars. In the other instances, well-known elements have not been shown or described to avoid unnecessarily obscuring the invention. Accordingly, the specification and drawings are to be regarded in an illustrative, rather that a restrictive, sense.

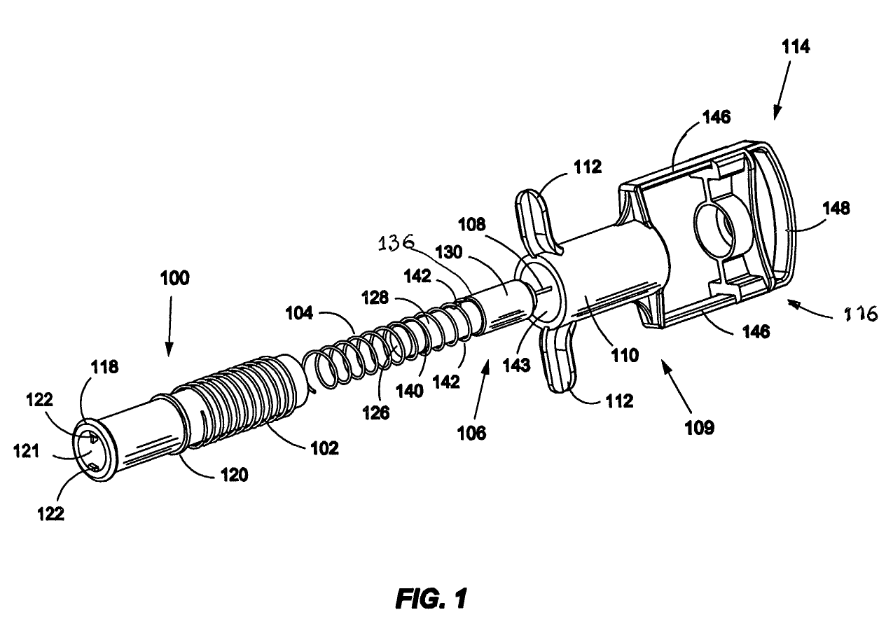

An exploded view of the lancet device according to one embodiment of the invention is illustrated in FIG. 1. The device includes a tube 100, springs 102 and 104, a lancet holder 106, a lancet 108, an outer member 109 comprising a hollow cylindrical body 110 integral with finger rests 112 at its trailing end and a loop 114 at its leading end, and a clamping member 116.

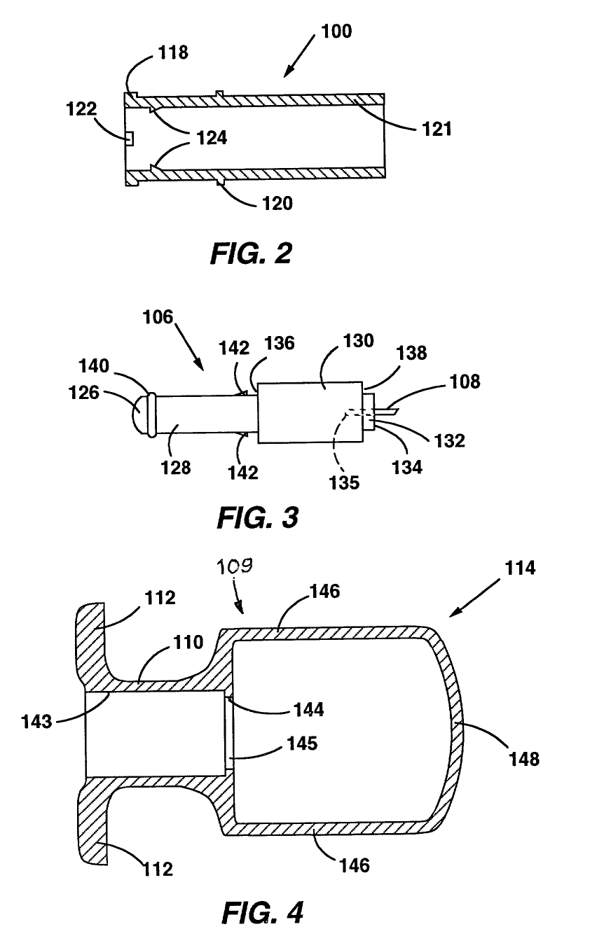

As shown in FIG. 2, tube 100 includes an external flange 118 at the trailing end thereof and a collar 120 located along the periphery of the tube. Tube 100 also has a bore 121 and a pair of diametrically-opposed projections 122 (only one of which is apparent from the drawing) formed on the interior of the tube at its trailing end. A pair of diametrically-opposed triangular barbs 124 is positioned on the interior of the tube.

The particulars of lancet holder 106 are discussed with reference to FIG. 3. The lancet holder has a convex end-face 126 at the trailing end thereof, a first cylindrical section 128, a second cylindrical section 130, a third cylindrical section 132, and a flat end-face 134 at the leading end of the lancet holder. The diameters of section 128 and 132 are substantially equal and are smaller than that of section 130. End-face 134 includes a cavity 135 in which lancet 108 is mounted in a conventional manner. Cylindrical section 130 is defined by a trailing shoulder 136 and a leading shoulder 138. Cylindrical section 128 incorporates an annular protuberance 140 and a pair of diametrically-opposed triangular barbs 142.

As illustrated in FIG. 4, hollow cylindrical body 110 of outer member 109 has a bore 143 and an interior flange 144 formed inside the bore at the leading end of the cylindrical body. Flange 144 defines an opening 145, whose diameter is less than the outer diameter of collar 120. Loop 114 comprises a pair of rails 146, bridged by an arcuate band 148.

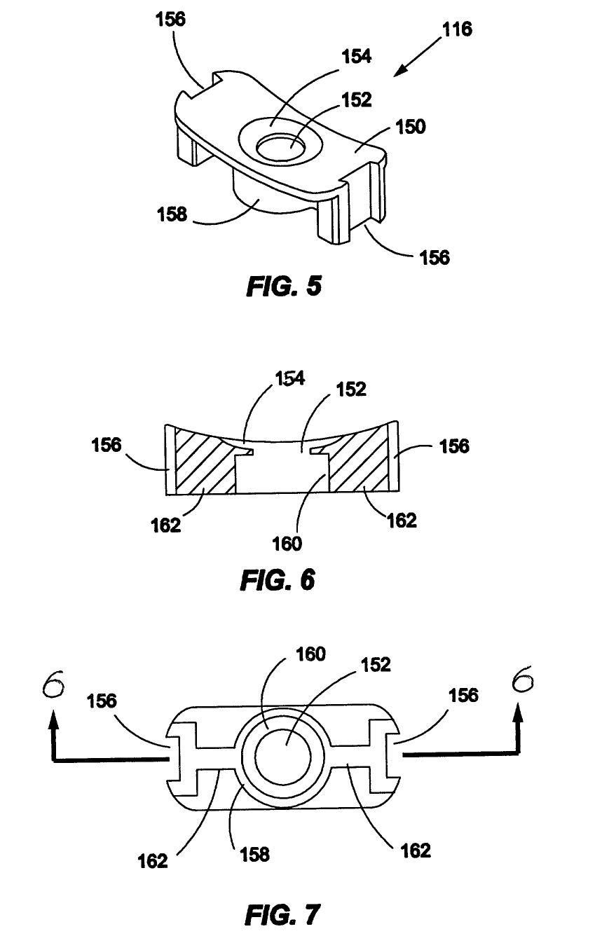

FIG. 5 is a perspective view of clamping member 116, which includes a curved platform 150 having a centrally-oriented through opening 152 and a countersink 154, concentric with opening 152. The diameter of opening 152 is smaller than the radial dimension of section 130 of the lancet holder, but has a clearance fit with section 132 thereof. The curvature of platfrom 150 substantially compliments that of a digit of a human hand. A pair of recesses 156 are provided on the opposite sides of platform 150. Member 116 also includes a cylindrical neck 158. FIG. 6, which is a sectional view of member 116, illustrates that neck 158 contains a counterbore 160, which communicates with opening 152. As apparent from FIG. 6 and also from FIG. 7 (bottom plan view of member 116), a pair of reinforcing members 162 are located between neck 158 and recesses 156 to enhance the rididity of the clamping member. Counter bore 160 is sized to make the outer surface of tube 100.

All the above-described components of the lancet device may be manufactured from injection-molded plastic, except for lancet 108, which is preferably made of stainless steel or other materials used for surgical appliances. It will be obvious to one of ordinary skill in the art that a variety of materials and manufacturing techniques may be utilized without departing from the scope of the present invention.

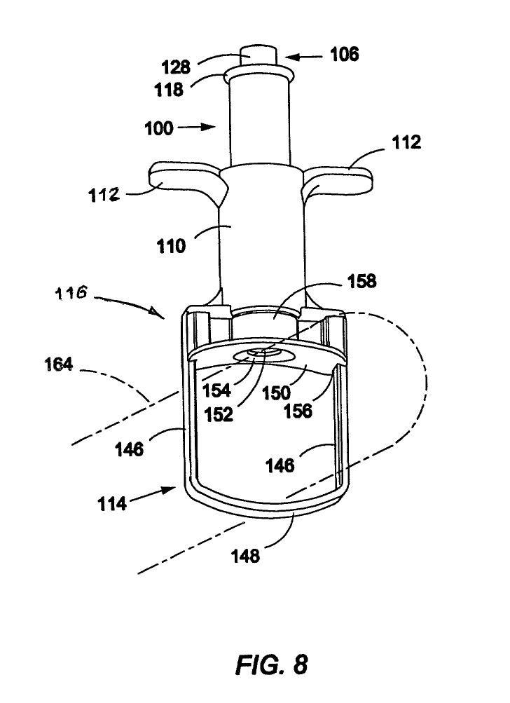

FIG. 8 is a perspective view of the assembled and armed lancet device of this embodiment. The assembly sequence of the lancet device is described below. As shown in FIG. 1, spring 104 is positined with respect to section 128 of lancet holder 106 such that the leading face of the spring seats against shoulder 136 (FIG. 3) and the leading coil of the spring engages the vertical face of one of barbs 142. A clearance fit exists between the helical coils of spring 104 and the surface of section 128. The radial distance between the tips of barbs 142 is less than the diameter of section 130 (FIG. 3), but produces an interference fit with the coils of spring 104. Therefore, the leading coil of spring 104 must expand sightly as it is forced past the barbs. The expansion of the coil is facilitated by the triangular shape of the barbs, oriented with the oblique surfaces toward the leading end of the spring. Once the leading coil clears the barb tips, it contracts, engaging the vertical face of one of the barbs. The outer diameter of protuberance 140 (FIG. 3) is less than the inner diameter of the helical coils of spring 104, allowing a clearance fit between the spring and protuberance 140.

The assembly comprising lancet holder 106 and spring 104 is then inserted into bore 121 of tube 100 from the leading end of the tube, with lancet 108 facing away from the bore. Cylindrical section 130 of the lancet holder has a sliding clearance fit with bore 121. Spring 104 also has a clearance fit therewith. At this point, the lancet device may be armed by advancing the holder/spring assembly into the bore of the tube, e.g., with an elongated pushrod (not shown) acting against end-face 134 (FIG. 3), until annular protuberance 140 of the lancet holder interlocks with projections 122 of the tube by virtue of an interference fit. (Alternatively, the arming of the lanced device may be performed after all assembly steps have been completed.) As the lancet holder/spring assembly is advanced into the tube, the trailing coil of spring 140 expands past the oblique portions of barbs 124 (FIG. 2) and engages the vertical face of one of the barbs, whereby the trailing face of the spring becomes seated against projections 122. Once protubrance 140 engages projections 122, the pushrod is withdrawn. The tube/lancet holder assembly remains locked in the armed position because the frictional force provided by the interference fit between protuberance 140 and projections 122 exceeds the force exerted by spring 104, which is compressed between projections 122 and shoulder 136. It should be noted that protuberance 140 has a clearance fit with triangular barbs 124.

Next, rails 146 of loop 114 are slightly deformed outward to allow recesses 156 of clamping member 116 to be positioned, one at a time, along the rails, so that member 116 is free to translate with respect to the rails of the loop. Spring 100 is then placed over the leading end of tube 100 so that it seats against collar 120 and the tube is inserted into the trailing end of the bore of hollow cylindrical body 110. Collar 120 of the tube has a sliding clearance fit with bore 143 and flange 144 (FIG. 4) has a sliding clearance fit with the peripheral surface of tube 100. The tube is then advanced into the bore until the leading end of the tube emerges through opening 145 (FIG. 4), causing spring 102 to be compressed between collar 120 of the tube and flange 144 of the hollow cylindrical body. The end of tube 100 protruding through opening 145 is then inserted into counterbore 160 of the clamping member and a coupling therebetween is acheived using conventional methods (e.g., a press-fit, adhesives, ultrasonic bonding, or other known techniques). The values of spring constants and lengths of springs 102 and 104, the distance between collar 120 and the leading end of tube 100, and the distance between protuberance 140 and shoulder 136 of the lancet holder are chosen such that even at maximum compression of spring 102 the combined force exerted by springs 102 and 104 is less than that of the friction force between protuberance 140 and projections 122.

The assembled and armed lancet device is shown in FIG. 8. In this configuration, spring 102 (not visible in FIG. 8) keeps tube 102 retracted with respect to cylindrical body 110, so that neck 158 of clamping member 116 is pressed against the outer surface defined by flange 144 of body 110. It should be understood that spring 102 is provided as a convenience feature only and is not necessary to the operation of the lancet device according to the invention, since tube 102 may be retraced with respect to cylindrical body 110 by other means, e.g., gravity.

As mentioned above, the lancet may alternatively be armed at this point, i.e., after assembly of all the component parts has been completed.

To collect a blood sample, a digit 164 of one hand (FIG. 8) is inserted into loop 114 while the patient, or operator, holds the lancet device in his or her other hand with the index and middle fingers covering rests 112 and the thumb pressed against end-face 126 of the lancet holder. The patient then uses the thumb to apply increasing pressure on the end-face 126, whereby tube 100 and clamping member 116 translate with respect to finger clamp 109 so that digit 164 is compressed between clamping member 116 and band 148 with increasing force. The tourniquet effect produced by the clamping member and the band on the digit of the patient causes the skin of the finger to swell, conforming to the contours of counter sink 154. Once the digit of the patient has been sufficiently squeezed between clamping member 116 and loop 114 to produce beneficial swelling of its tissue, additional pressure exerted on end-face 126 will overcome the frictional force between protuberance 140 of lancet holder 106 and projections 122 of tube 100, whereby lancet 108 is discharged and propelled by spring 104 toward the digit. Spring 104 is sized so that when the extension of the spring reached its maximum point, the lancet momentarily protrudes through opening 145 of body 110 and pierces the skin of the finger, producing an incision of a desired size and shape (in accordance with the size and shape of the lancet). As the incision is being made, the level of discomfort experienced by the user is minimized due to beneficial swelling of the tissues at the point of the incision, which prevents the lancet from striking too close to the bone of the digit. The above-mentioned swelling results from the tourniquet action produced by member 116 and loop 114 and is augmented by the presence of countersink 154, which promotes the swelling of the tissue directly around the point of the incision.

Upon making the incision, lancet 108 is permanently retracted into bore 143 by spring 104, which contracts to its equilibrium position. Spring 104, opposite ends of which remain attached to barbs 124 of tube 100 and barbs 124 of lancet holder 106, ensures that the tip of lancet 108 remain below the surface of platform 150. Since the construction of the lancet device is such that the instrument cannot be disassembled and re-armed (in its preferred use), the device may at this point be safely disposed without risking injury and infection to individuals who may come into contact with the lancet device.

Once the incision has been made, pressure on end-face 126 is released removing the compressive force of loop 114 and member 116 on the finger. The loop can then be moved farther back along the finger and pressure on the finger may be reapplied by once again pushing on end-face 126. The re-tightening of the clamping member and the loop around the finger causes blood to be forced out of the puncture previously made by the lancet, and allows multiple drops of blood to be collected for analysis.

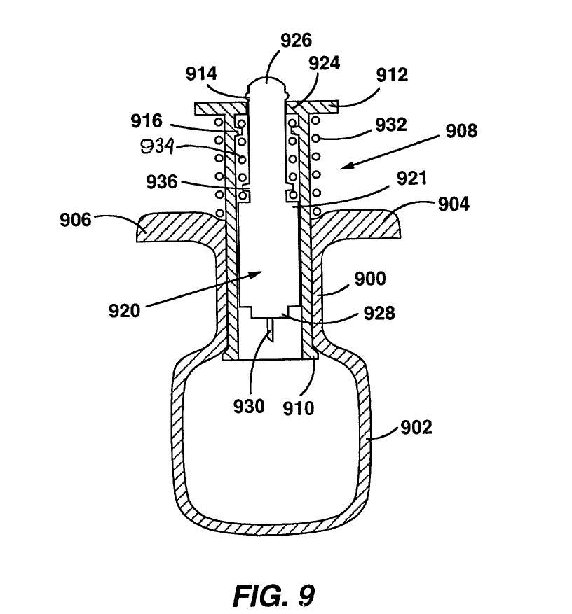

FIG. 9 is a sectional view through an alternative embodiment of the present invention. In this embodiment the outer sleeve 900 includes the means in the form of a loop 902 for restraining the finger or digit of the patient. An inner sleeve 908 is movably disposed relative to the outer sleeve 900 and within the outer sleeve. The inner sleeve 908 is coaxially interposed between the outer sleeve 900 and a depressible lanceted plunger 920. The lanceted plunger 920 is formed with outwardly projecting ears 914 and the inner sleeve is formed with inwardly projecting ribs 924 that cooperate with each other to restrain the plunger 920 in its armed position and to permit release of the plunger for movement within the inner sleeve 908, as will be described. The plunger 920 is formed with an upper shoulder 921 and a coil spring biasing means 934 engages the upper side of the shoulder 921 and the lower side of the projecting ribs 924 as a means for biasing the lanceted plunger 920 relative to the inner sleeve 908. A lancet 930 is attached to the lower end 928 of the plunger and extends the desired distance to accomplish the desired incision in a finger or digit.

The inner sleeve 908 is biased within the outer sleeve and away from the loop portion 902 when this embodiment is assembled. The lower end of the inner sleeve 908 includes outwardly extending ribs 910 the are constructed to engage the inner lower end of the outer sleeve 900 where the loop portion 902 attaches to the outer sleeve 900. These ribs 910 are designed to be flexible enough to pass into the inside of the outer sleeve and pass into the loop portion and them become stopped as the inner sleeve is biased out of the inner sleeve. The biasing force between the inner sleeve 908 and the outer sleeve 900 is provided by spring 932 that operates between the upper inner surface of the outer sleeve at the outwardly extending ribs 912 at the upper outer surface of the inner sleeve 908.

The lanceted plunger 920 is biased within the inner sleeve in a stored position by spring 934. The spring 934 provides the force needed to project the lanceted plunger to make an incision in a digit held within the loop 902. When the plunger is released, as will be described hereinafter, the spring forces the plunger through the inner sleeve. The plunger 920 is provided with an extending rib 936 spaced a small distance from the upper shoulder 921; this rib 936 engages the lowermost coil of the biasing spring 934 to return the plunger to a relaxed position within the inner sleeve 908 after the lancet has been forced from its cocked position. The upper coil of the biasing spring 934 is retained at the upper end of the inner sleeve 908 by engagement with ribs extending inwardly from the inner surface of the inner sleeve 908.

In operation of this embodiment of the invention, the assembly is as shown in FIG. 9 with the lanceted plunger 920 held in a cocked position by the engagement of the ears 914 with the ribs 924 and the bias of spring 934 prepared to force the plunger through the inside of the inner sleeve upon release of the ear/rib engagement. As the user grips the outer sleeve at the finger rests 904 and 906 and presses the inner sleeve downwardly into the outer sleeve 900, the lower end of the inner sleeve engages the digit captured within the loop 902 and creates the desired compression of the digit as has been previously described. Further force against the inner sleeve 908 by pressing on the extension 926 of the upper end of the plunger 920 causes the plunger to pass through the ear/rib engagement and to release the bias of the spring 934 to force the plunger 920 downwardly and to cause the lancet 930 to pierce the digit when at its fullest extension. After piercing the digit the bias of spring 934 returns the plunger to the stored position within the inner sleeve 908 and with the lancet end drawn into the sleeve. When the user's force is removed from forcing the inner sleeve into the outer sleeve, the bias of spring 932 returns the inner sleeve 908 into its withdrawn position within the outer sleeve 900. The lancet device is not reusable because the bias of spring 934 has been released and the plunger cannot be forced out of the inner sleeve 908.

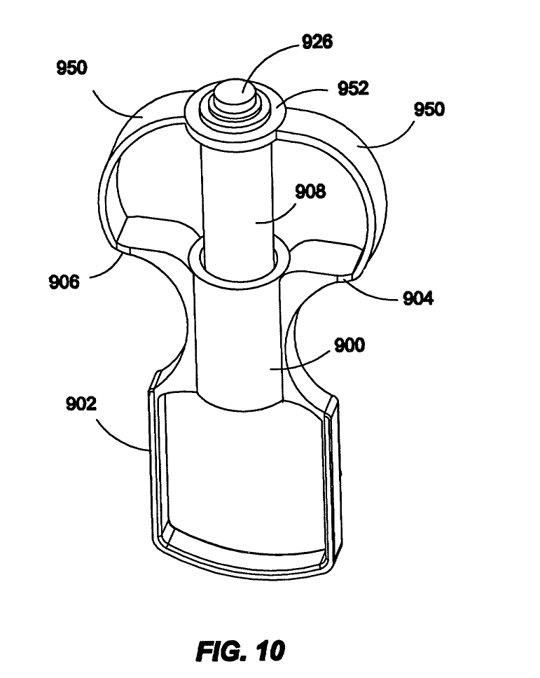

FIG. 10 illustrates an alternative form for the biasing means for the engagement of the inner sleeve 908 against a digit retainded within the loop 902. In this form, the bias between the outer sleeve 900 and the inner sleeve 908 is provided by a leaf spring like assembly 950 that engages the upper surface of the finger rests 904/906 at one end and the extension 926 of the inner sleeve 908. The internal bias of the lanceted plunger within the inner sleeve in this embodiment may be the same as that described with respect to FIG. 9 with the upper extension movement toward the outer sleeve 900 causing the lower end of the inner sleeve 908 to engage the digit within the loop and the eventual release of the lanceted plunger to make the incision in the digit. The inner sleeve is withdrawn as is the case in FIG. 9.

The leaf spring assembly includes a ring portion 952 that encloses the upper end of the extension 926. The extension has a first set of radially extending ears 954 near its upper end and a second set of extending ears 956 below the upper ears and along the body of th3 dextension 926. The ring portion passes over the first set of ears to place the lancet device in its position for use and over the lower set of ears after use. The upper ears cooperate with the ring portion 952 when the lancet device is in its cocked position and the lower ears cooperate with ring to return the inner sleeve to a stored position after use.

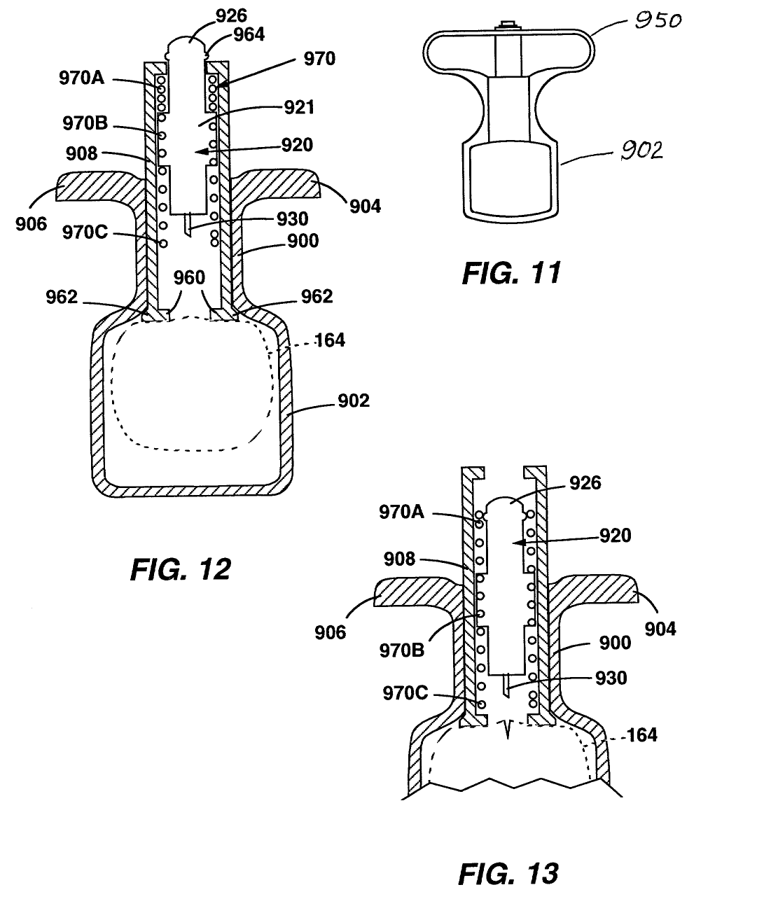

FIG. 11 illustrates the position of the leaf spring assembly prior to release of the inner sleeve and the lanceted plunger.

FIGS. 12 and 13 illustrate another alternative embodiment of the present invention. This embodiment uses a single spring for the force to send the lanceted plunger to its incision position and for return of the plunger and lancet to the stored positin. In these FIGs the outer sleeve 900 includes the finger or digit loop 902 (here shown as rectangular) and encloses the inner sleeve 908 in slideable engagement. The inner sleeve 908 has, at the lower end, inwardly extending ribs 960 and outwardly extending ribs 962 and, at the upper end, inwardly extending ribs 964. A lanceted plunger 920 is slideably enclosed within the inner sleeve 908.

The plunger has a lancet 930 extending from its lower end and has a operator extension 926 from its upper end.

A single biasing spring is operational within the inner sleeve to bias the lanceted plunger for release to create an incision and for return of the plunger to a stored position. The biasing spring 970 includes three integral portions. An upper portion 970A circles the plunger above the shouldered portion 921 and engages the lower edges of the ears 964 extending inwardly from the inner sleeve 908. The middle portion 970B circles and engages the shoulder portion of the plunger; the engagement may be in grooves in the exterior surface of the shouldered portion 921. The lower portion 970C circles the lower entension of the plunger and extends beyond the distance of the lancet extension from the plunger.

In use this embodiment is assembled with the biasing spring aligned with the three sections of the lancet plunger and that subassembly is inserted into the inner sleeve 908 with the extension 926 passing through the upper end of the inner sleeve and engaging its lower shoulders against the inwardly extending ears 964 of the inner sleeve 908. When in that position, the upper end of the spring 970A is compressed between the upper end of the shoulder 921 and the inner surface of the ears 964, the middle portion 970B of the spring is engaged with the middle portion of the shoulder 921 of the plunger 920, and lower end 970C of the spring 970 is circling the lower end of the plunger and the lancet 930. When ready for use, the lancet device is positioned with the finger of a patient within the restraining loop 902 and the operator engages the finger rests 904/906 and the extension 926 to move the inner sleeve into the engage position with the contained digit. Further depression of the extension 926 causes the upper end to pass into the inner sleeve 908 and to release the biasing force of the portion 970A of spring 970 thus causing th lanceted plunger to move rapidly toward the enclosed digit and to cause the lancet 930 to penetrate the digit causing the desired incision. After the incision has been made, the lower portion 970C of the spring 970 pushes against the inwardly extending ears 960 of the inner sleeve 908 and retrieves the lanceted plunger into a stored position in the inner sleeve. FIG. 13 illustrates the stored position of the lancet plunger 920 after the device has been used.

The foregoing has described several embodimnets of a lancet device for drawing blood samples. The lancet device includes an outer sleeve, means for restraining a digit or member of a patient relative to the outer sleeve, an inner sleeve movably disposed relative to the outer sleeve, a depressible lanceted plunger, the inner sleeve is cooaxially interposed between the outer sleeve and the lanceted plunger, means for releasably engaging the lanceted plunger with the inner sleeve, and means for biasing the lanceted plunger relative to the inner sleeve.

While certain preferred embodiments of the invention have been specifically disclosed, it should be understood that the invention is not limited thereto as many variation will be raedily apparent to those skilled in the art and the invention is to be given it broadest possible interpertation within the terms of the following claims.

We claim:

1. A lancet device for drawing blood from a target area of a digit, said lancet device comprising:
an outer sleeve;
means for constraining the digit relative to said outer sleeve;
an inner sleeve movably disposed relative to said outer sleeve;
a depressible lanceted plunger, said inner sleeve coaxially interposed between said outer sleeve and said lanceted plunger;
means for releasably engaging said lanceted plunger with said inner sleeve; and
means for biasing said lanceted plunger relative to said inner sleeve.

2. The lancet device of claim 1 wherein said means for biasing said lanceted plunger relative to said inner sleeve comprises means for propelling said lanceted plunger toward the target area for making an incision therein responsive to said lanceted plunger being depressibly disengaged from said inner sleeve and means for automatically retracting said lanceted plunger from the target area once the incision has been made.

3. The lancet device of claim 2 further including means for increasing blood supply to the target area, said means for increasing blood supply disposed at the distal end of said inner sleeve and responsive to the digit being compreseed between said means for constraining the digit and said inner sleeve.

4. The lancet device of claim 3 further including means for guiding said inner sleeve relative to said means for constraining the digit.

5. The lancet device of claim 4 further including means for gripping said outer sleeve.

6. The lancet device of claim 3 further including means for gripping said outer sleeve.

7. The lancet device of claim 2 further including means for biasing said inner sleeve relative to said outer sleeve.

8. The lancet device of claim 7 further including means for increasing blood supply to said target area, said means for increasing blood supply disposed at the distal end of said inner sleeve and responsive to the digit being compressed between said means for constraining the digit and said inner sleeve.

9. The lancet device of claim 8 further including means for guiding said inner sleeve relative to said means for constraining the digit.

10. The lancet device of claim 9 further including means for gripping said outer sleeve.

11. The lancet device of claim 8 further including means for gripping said outer sleeve.

12. A lancet device for drawing blood from a target area of a digit, said lancet device comprising:
an outer sleeve;
a digit-constraining member fixed relative to said outer sleeve;
an inner sleeve movably disposed relative to said outer sleeve;
a depressible lanceted plunger configured to releasably engage said inner sleeve, said inner sleeve coaxially interposed between said outer sleeve and said lanceted plunger; and
a first biasing element coupled to said lanceted plunger.

13. The lancet device of claim 12 wherein said first biasing element is adapted to propel said lanceted plunger toward the target area for making an incision therein responsive to said lanceted plunger being depressible disengaged form said inner sleeve, said first biasing element adapted to automatically retract said lanceted plunger from the target area once the incision has been made.

14. The lancet device of claim 13 wherein said first biasing element is coupled to said inner sleeve.

15. The lancet device of claim 13 further including at least one tissue-displacing element disposed at the distal end of said inner sleeve, said at least one tissue-displacing element configured to increase blood supply to the target area responsive to the digit being compressed between said digit-constraining member and said inner sleeve.

16. The lancet device of claim 15 further including a guide fixed relative to said inner sleeve, said guide movably engaging said digit-constraining member.

17. The lancet device of claim 15 further including at least one grip fixed relative to said outer sleeve.

18. The lancet device of claim 15 further including at least one grip fixed relative to said outer sleeve.

19. The lancet device o f claim 13 further including a second biasing element longitudinally interposed between said outer sleeve and said inner sleeve.

20. The lancet device of claim 19 wherein said second biasing element is coupled to said inner sleeve.

21. The lancet device of claim 19 further including at least one tissue-displacing element disposed at the distal end of said inner sleeve, said at least one tissue-displacing element configured to increase blood supply to the target area responsive to the digit being compressed between said digit-constraining member and said inner sleeve.

22. The lancet device of claim 21 further including a guide fixed relative to said inner sleeve, said guide movably engaging said digit-constraining member.

23. The lancet device of claim 22 further including at least one grip fixed relative to said outer sleeve.

24. The lancet device of claim 21 further including at least one grip fixed relative to said outer sleeve.
